(19) United States
(12) Patent Application Publication
(10) Pub. No.: US 2022/0040279 A1
(43) Pub. Date: Feb. 10, 2022

(54) NEOANTIGEN COMPOSITIONS; AND METHODS OF PREPARATION AND USE THEREOF

(71) Applicant: THOMAS JEFFERSON UNIVERSITY, Pholadelphia, PA (US)

(72) Inventors: Douglas Craig Hooper, Medford, NJ (US); Samantha Garcia, Philadelphia, PA (US)

(21) Appl. No.: 17/280,246

(22) PCT Filed: Sep. 26, 2019
(86) PCT No.: PCT/US2019/053102

§ 371 Date: Mar. 26, 2021

Related U.S. Application Data
(60) Provisional application No. 62/736,861, filed on Sep. 26, 2018.

(51) Int. Cl. A61K 39/00; C12N 15/113; C12N 15/117

(52) U.S. Cl. CPC A61K 39/001103; C12N 15/1138; C12N 2310/17; A61K 2039/80; C12N 2310/11; C12N 15/117

(57) ABSTRACT

The disclosure provides methods of producing neoantigens, comprising bringing a tumor cell culture in contact with an antisense oligonucleotide; and further, recovering the neoantigens; as well as immunogenic compositions comprising these neoantigens. The disclosure further provides methods of inducing an immune response in a subject against a cancer such as glioblastoma; and methods of treating a cancer such as glioblastoma in a subject comprising administering to the subject a therapeutically effective amount of the neoantigens and compositions disclosed herein.

### CROSS REFERENCE TO RELATED APPLICATIONS

This application claims the benefit under 35 U.S.C. § 119(e) to U.S. Provisional Patent Application No. 62/736,861, filed Sep. 26, 2018, the contents of which are incorporated herein by reference in its entirety for all purposes.

### FIELD

The present disclosure relates to compositions and methods for treating or immunizing against cancers using antigens that are derived from cancer cells.

### INCORPORATION BY REFERENCE OF SEQUENCE LISTING

The contents of the text file named “1MVX_013_01WO_SeqList_ST25”, which was created on Sep. 23, 2019 and is 9.42 kilobytes in size, are hereby incorporated by reference in its entirety.

### BACKGROUND

Despite advances in cancer therapy, the prognosis for malignant glioma, particularly glioblastoma multiforme, and many other cancers remains poor. Modifications of standard treatments such as, for example, chemotherapy, external beam radiation, and brachytherapy provide only small increments of improvement in both progression-free survival and overall survival. Immunotherapy trials, although promising in theory, have not addressed the challenges created by solid tumors. For the treatment of glioma, the National Cancer Institute estimates an annual incidence of around 28,000 cases annually, which increases to over 50,000 if patients with recurrent gliomas are included. Therefore, there is a need in the art to obtain new and improved treatments for cancers, and cancers of the brain in particular.

### SUMMARY

Disclosed herein are methods of producing neoantigens, comprising bringing a tumor cell culture in contact with an antisense oligonucleotide, wherein the tumor cell culture is irradiated before or after bringing the culture in contact with the antisense oligonucleotide; and further, recovering the neoantigens. The disclosure also provides immunogenic compositions comprising the neoantigens prepared by the methods disclosed herein. Also disclosed herein, are methods of inducing an immune response in a subject; methods of inducing resistance to growth of a cancer in a subject; methods of inducing regression of a cancer in a subject; and methods of treating a cancer in a subject comprising administering to the subject a therapeutically effective amount of the neoantigens and compositions disclosed herein.

In particular aspects that antisense and irradiation treatments are performed in a container, such as a chamber containing a membrane with pores. The pores allow neoantigens to exit the chamber for use in immunogenic compositions. In other aspects, the remaining contents of the chamber may be used as neoantigens; in some cases, after a clarifying step, such as by using centrifugation. Conveniently, the neoantigens may be stored (e.g., frozen or lyophilized) and may be used as an initial therapy or as a boost therapy following other cancer therapies.

Other objects, features and advantages of the present disclosure will become apparent from the following detailed description. It should be understood, however, that the detailed description and the specific examples, while indicating preferred embodiments, are given by way of illustration only, since various changes and modifications within the spirit and scope of the disclosure will become apparent to those skilled in the art from this detailed description.

### BRIEF DESCRIPTION OF FIGURES

FIG. 1 shows the activation of immunized T-cells by neoantigens produced using irradiated 10⁶ GL261 cells, treated as described in Example 2 (Groups 1-7).

FIG. 2 shows the effect of the amount of NOBEL antisense used on the production of neoantigens. Example 3 describes the different diffusion chambers that were prepared and tested in the experiment (Groups 1-6, represented in Bars 1-6).

FIG. 3 shows the effect of pre-treating tumor cells with NOBEL antisense on the production of neoantigens. Example 4 describes the two diffusion chambers that were prepared and tested in the experiment (Groups 1 and 2, represented in Bars 1 and 2).

FIG. 4 shows the efficient production of glioma neoantigens from mouse glioma cells. Biodiffusion chambers were prepared as described in Example 5 (Groups 1-5). Chamber contents were then incubated with bone marrow-derived DC and the DC incubated overnight with T cells from GL261-immune mice. T cells were then recovered and the number producing IFN-γ determined in ELISPOTs. Data is presented as spots per CD4 T cell.

FIG. 5 shows the increase in IFN-γ producing T cells in response to neoantigens prepared using a primary human GBM cell line and NOBEL, under different conditions as described in Example 6 and indicated on the graph. The graph represents mean IFN-γ spots per 100,000 T cells and 10,000 DC with or without antigen +/− standard deviation. Multiple comparison of the background (DC+Tcells and DC+respective Antigen) with the response (DC+Tcells with respective antigen) is shown in the table.

### DETAILED DESCRIPTION

### Definitions

As used herein, terms such as “a,” “an,” and “the” include singular and plural referents unless the context clearly demands otherwise.

As used herein, the term “about” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, the terms “neoantigen,” “cancer neoantigen,” “tumor-specific antigen” and “tumor antigen” refer to antigens that are derived from cancer cells.

As used herein, the term “adjuvant” refers to a compound that, when used in combination with an antigen, augments or otherwise modifies the immune response induced against the antigen. Modification of the immune response may include intensification or broadening the specificity of either or both antibody and cellular immune responses.

The terms “cancer” and “tumor,” which are used interchangeably herein, refer to an uncontrolled division of abnormal cells in the body of a subject.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of cancer; ameliorating, or reducing the development of symptoms of cancer; or a combination thereof.

“Prevention” as used herein, is used interchangeably with “prophylaxis” and can mean complete prevention of a disease such as cancer, or prevention of the development of symptoms of that disease; a delay in the onset of that disease or its symptoms; or a decrease in the severity of a subsequently developed disease or its symptoms.

As used herein, an “effective dose” or “effective amount” refers to an amount of substance able to achieve a desired outcome; for example, an amount of an immunogen sufficient to induce an immune response that inhibits or suppresses the initiation or progression of cancer; ameliorates, or reduces the development of symptoms of cancer; or a combination thereof.

As used herein, an “immunogenic composition” or “vaccine” is a composition that comprises an antigen, such as a neoantigen, where administration of the composition to a subject results in the development in the subject of a humoral and/or a cellular immune response to the antigen.

As used herein, the term “subject” includes humans and other animals. Typically, the subject is a human. For example, the subject may be an adult, a teenager, a child (2 years to 14 years of age), an infant (1 month to 24 months), or a neonate (up to 1 month). In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

As used herein, the term “healthy subject” refers to a subject not suffering from cancer and not in need of treatment with the methods disclosed herein.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans.

### Methods of Producing Neoantigens

Cancer growth may be limited by an immune response to antigens produced by cancer cells. One traditional approach to inducing cancer-specific immunity in a subject is to use cancer cells for vaccination. In some instances, tumor cells modified to be more immunogenic, or dendritic cells pulsed with tumor cells may be used for vaccination. However, these tumor cells produce a variety of agents including proteins and RNAs that interfere with the induction of an effective immune response. Consequently, using intact tumor cells or viable tumor cells as tumor antigens may have disadvantages. Therefore, the traditional approaches for production of tumor antigens are inadequate for the induction of an effective immune response against cancer in subjects.

Disclosed herein are methods of producing neoantigens in vitro. The neoantigens produced by the methods disclosed herein induce an effective immune response against cancer. While intact tumor cells used in the traditional immunization approach described above have a short shelf life, the compositions comprising neoantigens disclosed herein have the potential to be stored for extended periods of time, which is especially advantageous for booster immunization.

In certain aspects, the methods of producing neoantigens disclosed herein comprises contacting tumor cells with an antisense oligonucleotide, wherein the culture is irradiated before and/or after contacting the cells with the antisense oligonucleotide, to produce neoantigens; and further, recovering the neoantigens.

In some aspects, the tumor cells remain in contact with the antisense oligonucleotide for at least about 1 hour, 2 hours, 4 hours, 6 hours, 8 hours, 12 hours, 18 hours or 24 hours. In certain aspects, the tumor cells are in contact with the antisense oligonucleotide for about 1 hour to about 24 hours, including all values and subranges that lie therebetween. For example, the tumor cells may be in contact with the antisense oligonucleotide for about 4 hours, 12 hours or 18 hours. In some aspects the tumor cells are in contact with the antisense oligonucleotide for about 18 hours.

In some aspects, the tumor cells remain in contact with the antisense oligonucleotide at a temperature of about 10° C. to about 40° C., for example, about 15° C., about 18° C., about 20° C., about 22° C., about 24° C., about 26° C., about 28° C., about 30° C., about 32° C., about 34° C., about 36° C., about 37° C., about 38° C., about 39° C. or about 40° C., including all values and subranges that lie therebetween.

The irradiation of the tumor cells may be performed before and/or after bringing the tumor cells in contact with the antisense oligonucleotide. In some aspects, irradiation may be performed about 24 hours to about 5 min (including all values and subranges that lie therebetween) before bringing the tumor cells in contact with the antisense oligonucleotide. For example, irradiation may be performed before about 24 hours, about 12 hours, about 6 hours, about 5 hours, about 4 hours, about 3 hours, about 2 hours, about 1 hour, about 30 minutes, about 5 minutes or immediately before bringing the tumor cells in contact with the antisense oligonucleotide. In some aspects, irradiation may be performed about 24 hours to about 5 min (including all values and subranges that lie therebetween) after bringing the tumor cells in contact with the antisense oligonucleotide. For example, irradiation may be performed about 24 hours, about 12 hours, about 6 hours, about 5 hours, about 4 hours, about 3 hours, about 2 hours, about 1 hour, about 30 minutes, about 5 minutes or immediately after bringing the tumor cells in contact with the antisense oligonucleotide.

In certain aspects, the tumor cells are treated with gamma irradiation at an amount of about 1 Gy to about 15 Gy; preferably, at an amount of about 2 Gy to about 10 Gy. In certain aspects, the tumor cells are treated with gamma irradiation at an amount of about 1 Gy to about 200 Gy, for example about 10 Gy, about 20, about 30 Gy, about 40, about 50, about 60 Gy, about 70 Gy, about 80 Gy, about 90 Gy, about 100 Gy, about 125 Gy, about 150 Gy, about 175 Gy, or about 200 Gy, including all values and subranges that lie therebetween. In some aspects, the tumor cells are treated with gamma irradiation at an amount of about 1 Gy, about 2 Gy, about 4 Gy, about 5 Gy, about 6 Gy, about 10 Gy, or up to about 15 Gy. In certain aspects, the dose of radiation is not more than about 5 Gy. In other aspects, the dose of radiation is at least about 5 Gy. In some aspects, the dose of radiation is 5 Gy. In certain aspects, the cell culture may be irradiated at least once, at least twice, at least three times, at least four times, or at least five times.

In some aspects, the methods of producing neoantigens disclosed herein may comprise additional steps, as needed for recovering the neoantigens, such as centrifugation, filtration, sterilization, lyophilization, freezing or a combination thereof. Thus, in particular aspects, the disclosure provides lyophilized or frozen combinations containing neoantigens.

In some aspects, the neoantigens are recovered using centrifugation. As shown in the Examples, the neoantigens produced by the cells may comprise different sizes. Some neoantigens diffuse through the pores of a container (e.g., diffusion chamber pore), while other neoantigens are too large. In some aspects, the neoantigen recovery includes recovering just the neoantigens that can diffuse through the pores of a chamber, for example, neoantigens having a volume less than 100 μm³. In some aspects, the neoantigen recovery includes recovering just the neoantigens that cannot diffuse through the pores of the chamber, for example, neoantigens having a volume more than the 100 μm³. In some aspects, the neoantigen recovery includes recovering both the neoantigens that can diffuse through the pores in the chamber, for example, neoantigens having a volume less than 100 μm³; and neoantigens that cannot diffuse through the pores of the chamber, for example, neoantigens have a volume more than the 100 μm³. In embodiments, the neoantigen compositions disclosed herein are cell-free, and free of cellular debris. Cell-free compositions may be obtained by pelleting the cells and cell debris using centrifugation, and recovering the supernatant. In aspects, low-speed centrifugation is used to pellet the cells. In aspects, high-speed centrifugation is used to pellet the cell debris. In aspects, the neoantigens are recovered using discontinuous gradient centrifugation and/or chromatographic techniques.

Thus, in aspects, the neoantigens may be recovered by any combination of one or more biochemical purification steps disclosed herein, and/or one or more biochemical purification steps known in the art. The combination of biochemical purification steps used may depend on the size of the neoantigen being recovered. In aspects, a combination of the biochemical purification steps disclosed herein may be used to recover neoantigens that have a volume more than the 100 μm³. In aspects, a combination of the biochemical purification steps disclosed herein may be used to recover neoantigens that have a volume less than the 100 μm³.

### Antisense Oligonucleosides

The antisense oligonucleotides disclosed herein targets the Insulin like Growth Factor 1 Receptor (IGF-1R). IGF-1R is a tyrosine kinase cell surface receptor that shares 70% homology with the insulin receptor. When activated by its ligands (IGF-I, IGF-II and insulin), it regulates broad cellular functions including proliferation, transformation and cell survival. The IGF-1R is not an absolute requirement for normal growth, but it is essential for growth in anchorage-independent conditions that may occur in malignant tissues. A review of the role of IGF-IR in tumors is provided in Baserga el al., Vitamins and Hormones, 53:65-98 (1997), which is incorporated herein by reference in its entirety.

The full length coding sequence of IGF-1R is provided as SEQ ID NO: 1 and shown below (see, for example, PCT/US2016/26970, which is incorporated herein by reference in its entirety).

5′ATGAAGTCTGGCTCCGGAGGAGGGTCCCCGACCTCGCTGTGG
GGGCTCCTGTTTCTCTCCGCCGCGCTCTCGCTCTGGCCGACGAG
TGGAGAAATCTGCGGGCCAGGCATCGACATCCGCAACGACTAT
CAGCAGCTGAAGCGCCTGGAGAACTGCACGGTGATCGAGGGCT
ACCTCCACATCCTGCTCATCTCCAAGGCCGAGGACTACCGCAGC
TACCGCTTCCCCAAGCTCACGGTCATTACCGAGTACTTGCTGCT
GTTCCGAGTGGCTGGCCTCGAGAGCCTCGGAGACCTCTTCCCCA
ACCTCACGGTCATCCGCGGCTGGAAACTCTTCTACAACTACGCC
CTGGTCATCTTCGAGATGACCAATCTCAAGGATATTGGGCTTTA
CAACCTGAGGAACATTACTCGGGGGGCCATCAGGATTGAGAAA
AATGCTGACCTCTGTTACCTCTCCACTGTGGACTGGTCCCTGAT
CCTGGATGCGGTGTCCAATAACTACATTGTGGGGAATAAGCCC
CCAAAGGAATGTGGGGACCTGTGTCCAGGGACCATGGAGGAGA
AGCCGATGTGTGAGAAGACCACCATCAACAATGAGTACAACTA
CCGCTGCTGGACCACAAACCGCTGCCAGAAAATGTGCCCAAGC
ACGTGTGGGAAGCGGGCGTGCACCGAGAACAATGAGTGCTGCC
ACCCCGAGTGCCTGGGCAGCTGCAGCGCGCCTGACAACGACAC
GGCCTGTGTAGCTTGCCGCCACTACTACTATGCCGGTGTCTGTG
TGCCTGCCTGCCCGCCCAACACCTACAGGTTTGAGGGCTGGCGC
TGTGTGGACCGTGACTTCTGCCAACATCCTCAGCGCCGAGAGC
AGCGACTCCGAGGGGTTTGTGATCCACGACGGCGAGTGCATGC
AGGAGTGCCCCTCGGGCTTCATCCGCAACGGCAGCCAGAGCAT
GTACTGCATCCCTTGTGAAGGTCCTTGCCCGAAGGTCTGTGAGG
AAGAAAAGAAAACAAAGACCATTGATTCTGTTACTTCTGCTCA
GATGCTCCAAGGATGCACCATCTTCAAGGGCAATTTGCTCATTA
ACATCCGACGGGGGAATAACATTGCTTCAGAGCTGGAGAACTT
CATGGGGCTCATCGAGGTGGTGACGGGCTACGTGAAGATCCGC
CATTCTCATGCCTTGGTCTCCTTGTCCTTCCTAAAAAACCTTCGC
CTCATCCTAGGAGAGGAGCAGCTAGAAGGGAATTACTCCTTCT
ACGTCCTCGACAACCAGAACTTGCAGCAACTGTGGGACTGGGA
CCACCGCAACCTGACCATCAAAGCAGGGAAAATGTACTTTGCT
TTCAATCCCAAATTATGTGTTTCCGAAATTTACCGCATGGAGGA
AGTGACGGGGACTAAAGGGCGCCAAAGCAAAGGGGACATAAA
CACCAGGAACAACGGGGAGAGAGCCTCCTGTGAAAGTGACGTC
CTGCATTTCACCTCCACCACCACGTCGAAGAATCGCATCATCAT
AACCTGGCACCGGTACCGGCCCCTGACTACAGGGATCTCATCA
GCTTCACCGTTTACTACAAGGAAGCACCCTTTAAGAATGTCACA
GAGTATGATGGGCAGGATGCCTGCGGCTCCAACAGCTGGAACA
TGGTGGACGTGGACCTCCCGCCCAACAAGGACGTGGAGCCCGG
CATCTTACTACATGGGCTGAAGCCCTGGACTCAGTACGCCGTTT
ACGTCAAGGCTGTGACCCTCACCATGGTGGAGAACGACCATAT
CCGTGGGGCCAAGAGTGAGATCTTGTACATTCGCACCAATGCTT
CAGTTCCTTCCATTCCCTTGGACGTTCTTTCAGCATCGAACTCCT
CTTCTCAGTTAATCGTGAAGTGGAACCCTCCCTCTCTGCCCAAC
GGCAACCTGAGTTACTACATTGTGCGCTGGCAGCGGCAGCCTC
AGGACGGCTACCTTTACCGGCACAATTACTGCTCCAAAGACAA
AATCCCCATCAGGAAGTATGCCGACGGCACCATCGACATTGAG
GAGGTCACAGAGAACCCCAAGACTGAGGTGTGTGGTGGGGAGA
AAGGGCCTTGCTGCGCCTGCCCCAAAACTGAAGCCGAGAAGCA
GGCCGAGAAGGAGGAGGCTGAATACCGCAAAGTCTTTGAGAAT
TTCCTGCACAACTCCATCTTCGTGCCCAGACCTGAAAGGAAGCG
GAGAGATGTCATGCAAGTGCAACACCACCATGTCCAGCCGAAG
CAGGAACACCACGGCCGCAGACACCTACAACATCACCGACCCG
GAAGAGCTGGAGACAGAGTACCCTTTCTTTGAGAGCAGAGTGG
ATAACAAGGAGAGAACTGTCATTTCTAACCTTCGGCCTTTCACA
TTGTACCGCATCGATATCCACAGCTGCAACCACGAGGCTGAGA
AGCTGGGCTGCAGCGCCTCCAACTTCGTCTTTGCAAGGACTATG
CCCGCAGAAGGAGCAGATGACATTCCTGGGCCAGTGACCTGGG
AGCCAAGGCCTGAAAACTCCATCTTTTTAAAGTGGCCGGAACCT
GAGAATCCCAATGGATTGATTCTAATGTATGAAATAAAATACG
GATCACAAGTTGAGGATCAGCGAGAATGTGTGTCCAGACAGGA
ATACAGGAAGTATGGAGGGGCCAAGCTAAACCGGCTAAACCCG
GGGAACTACACAGCCCGGATTCAGGCCACATCTCTCTCTGGGA
ATGGGTCGTGGACAGATCCTGTGTTCTTCTATGTCCAGGCCAAA
ACAGGATATGAAAACTTCATCCATCTGATCATCGCTCTGCCCGT
CGCTGTCCTGTTGATCGTGGGAGGGTTGGTGATTATGCTGTACG
TCTTCCATAGAAAGAGAAATAACAGCAGGCTGGGGAATGGAGT
GCTGTATGCCTCTGTGAACCCGGAGTACTTCAGCGCTGCTGATG
TGTACGTTCCTGATGAGTGGGAGGTGGCTCGGGAGAAGATCAC
CATGAGCCGGGAACTTGGGCAGGGGTCGTTTGGGATGGTCTAT
GAAGGAGTTGCCAAGGGTGTGGTGAAAGATGAACCTGAAACCA
GAGTGGCCATTAAAACAGTGAACGAGGCCGCAAGCATGCGTGA
GAGGATTGAGTTTCTCAACGAAGCTTCTGTGATGAAGGAGTTCA
ATTGTCACCATGTGGTGCGATTGCTGGGTGTGGTGTCCCAAGGC
CAGCCAACACTGGTCATCATGGAACTGATGACACGGGGCGATC
TCAAAAGTTATCTCCGGTCTCTGAGGCCAGAAATGGAGAATAA
TCCAGTCCTAGCACCTCCAAGCCTGAGCAAGATGATTCAGATG
GCCGGAGAGATTGCAGACGGCATGGCATACCTCAACGCCAATA
AGTTCGTCCACAGAGACCTTGCTGCCCGGAATTGCATGGTAGCC
GAAGATTTCACAGTCAAAATCGGAGATTTTGGTATGACGCGAG
ATATCTATGAGACAGACTATTACCGGAAAGGAGGGAAAGGGCT
GCTGCCCGTGCGCTGGATGTCTCCTGAGTCCCTCAAGGATGGAG
TCTTCACCACTTACTCGGACGTCTGGTCCTTCGGGGTCGTCCTCT
GGGAGATCGCCACACTGGCCGAGCAGCCCTACCAGGGCTTGTC
CAACGAGCAAGTCCTTCGCTTCGTCATGGAGGGCGGCCTTCTGG
ACAAGCCAGACAACTGTCCTGACATGCTGTTTGAACTGATGCGC
ATGTGCTGGCAGTATAACCCCAAGATGAGGCCTTCCTTCCTGGA
GATCATCAGCAGCATCAAAGAGGAGATGGAGCCTGGCTTCCGG
GAGGTCTCCTTCTACTACAGCGAGGAGAACAAGCTGCCCGAGC
CGGAGGAGCTGGACCTGGAGCCAGAGAACATGGAGAGCGTCC
CCCTGGACCCCTCGGCCTCCTCGTCCTCCCTGCCACTGCCCGAC
AGACACTCAGGACACAAGGCCGAGAACGGCCCCGGCCCTGGG
GTGCTGGTCCTCCGCGCCAGCTTCGACGAGAGACAGCCTTACG
CCCACATGAACGGGGGCCGCAAGAACGAGCGGGCCTTGCCGCT
GCCCCAGTCTTCGACCTGCTGA-3′

In certain aspects, the antisense oligonucleotide comprises nucleic acid sequences complementary to the nucleic acid sequence encoding the IGF-1R signal sequence. The signal sequence of IGF-1R is a 30 amino acid sequence. In some aspects, the antisense oligonucleotide comprises nucleotide sequences complementary to portions of the nucleic acid sequence encoding the IGF-1R signal sequence. In some aspects, the antisense oligonucleotide comprises nucleotide sequences complementary to codons 1-309 of IGF-1R, or portions thereof.

In some aspects, the antisense oligonucleotide comprises the nucleotide sequence of SEQ ID NO: 2, or a fragment thereof. In certain aspects, the antisense oligonucleotide comprises at least about 70%, at least about 75%, at least about 80%, at least about 85%, at least about 90%, at least about 95%, at least about 96%, at least about 97%, at least about 98%, at least about 99%, or 100% identity to SEQ ID NO: 2, or a fragment thereof.

In certain aspects, the antisense oligonucleotide consists of SEQ ID NO: 2. In some aspects, the antisense oligonucleotide is NOBEL, which has the sequence of SEQ ID NO: 2, NOBEL antisense as used herein has a fully phosphorothioate backbone, unless noted otherwise. The NOBEL sequence, derived as the complementary sequence of the IGF-1R gene at the 5′ end, is:

5′-TCCTCCGGAGCCAGACTT-3′.

NOBEL has a stable shelf life and is resistant to nuclease degradation due to its phosphorothioate backbone. The 18-mer NOBEL sequence has both IGF-1R receptor downregulation activity as well as TLR agonist activity. These activities might contribute to its in vivo anti-tumor immune activity.

In certain aspects, the sequence of the antisense oligonucleotide is selected from the group consisting of SEQ ID Nos. 2-15, as shown in Table 1. In some aspects, the antisense oligonucleotide comprises at least about 70%, at least about 75%, at least about 80%, at least about 85%, at least about 90%, at least about 95%, at least about 96%, at least about 97%, at least about 98%, at least about 99%, or 100% identity to SEQ ID Nos. 2-15, or a fragment thereof.

TABLE 1

| | Corresponds to | SEQ |
|---|---|---|
| Sequences with ACGA Motif | IGF-1R Codons | ID NO: |
| 5’-TCCTCCGGAGCCAGACTT-3’ | 2-7 | 2 |
| 5’-TTCTCCACTCGTCGGCC-3’ | 26-32 | 3 |
| 5’-ACAGGCCGTGTCGTTGTC-3’ | 242-248 | 4 |
| 5’-GCACTCGCCGTCGTGGAT-3’ | 297-303 | 5 |
| 5’-CGGATATGGTCGTTCTCC-3’ | 589-595 | 6 |
| 5’-TCTCAGCCTCGTGGTTGC-3’ | 806-812 | 7 |
| 5’-TTGCGGCCTCGTTCACTG-3’ | 1,033-1,039 | 8 |
| 5’-AAGCTTCGTTGAGAAACT-3’ | 1,042-1,048 | 9 |
| 5’-GGACTTGCTCGTTGGACA-3’ | 1,215-1,221 | 10 |
| 5’-GGCTGTCTCTCGTCGAAG-3’ | 1,339-1,345 | 11 |
| 5’-CAGATTTCTCCACTCGTCGG-3’ | 27-34 | 12 |
| 5’-CCGGAGCCAGACTTCAT-3’ | 1-6 | 13 |
| 5’-CTGCTCCTCCTCTAGGATGA-3’ | 407-413 | 14 |
| 5’-CCCTCCTCCGGAGCC-3’ | 4-8 | 15 |

In some aspects, the antisense oligonucleotide is a DNA molecule. In some aspects, the antisense oligonucleotide is an RNA molecule. In certain aspects, the antisense oligonucleotide is at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, or at least about 50 nucleotides in length. In some aspects, the antisense oligonucleotide is from about 5 nucleotides to about 50 nucleotides in length; preferably, from about 15 nucleotides to about 25 nucleotides in length. In certain aspects, the antisense oligonucleotide is about 18 nucleotides in length.

In some aspects, the antisense oligonucleotides comprise a modified phosphate backbone. In certain aspects, the phosphate backbone modification renders the antisense oligonucleotide more resistant to nuclease degradation. In certain aspects, the antisense oligonucleotide is a locked antisense oligonucleotide.

The antisense oligonucleotide, for example the NOBEL sequence of SEQ ID NO: 2, may comprise one or more p-ethoxy backbone modifications as disclosed in U.S. Pat. No. 9,744,187, which is incorporated by reference herein in its entirety. In some aspects, the nucleic acid backbone of the antisense oligonucleotide comprises at least one p-ethoxy backbone linkage. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% of the antisense oligonucleotide may be p-ethoxy-linked. For an 18-mer such as the NOBEL sequence, the number of p-ethoxy links may be at least 2, 3, 4, 5, 6, 7, 8, 9, 10, 11, 12, 13, 14, 15, or 16. In some cases, all linkages may be p-ethoxy but typically this is not the case and for NOBEL.

In some aspects, the modification is a phosphorothioate linkage. In certain aspects, the antisense oligonucleotide contains one or more phosphorothioate linkages. In certain aspects, the phosphorothioate linkages stabilize the antisense oligonucleotide by conferring nuclease resistance, thereby increasing its half-life. In some aspects, the antisense oligonucleotide may be partially phosphorothioate-linked. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% of the antisense oligonucleotide may be phosphorothioate-linked. In some aspects, the antisense oligonucleotide is fully phosphorothioate-linked. In other aspects, phosphorothioate linkages may alternate with phosphodiester linkages. In certain aspects, the antisense oligonucleotide has at least one terminal phosphorothioate monophosphate.

In some aspects, the antisense oligonucleotide does not comprise a CpG motif. In some aspects, the antisense oligonucleotide comprises one or more CpG motifs. In certain aspects, the one or more CpG motifs are methylated. In other aspects, the one or more CpG motifs are unmethylated.

In certain aspects, the antisense oligonucleotide comprises at least one terminal modification or “cap”. The cap may be a 5′ and/or a 3′-cap structure. The terms “cap” or “end-cap” include chemical modifications at either terminus of the oligonucleotide (with respect to terminal ribonucleotides), and including modifications at the linkage between the last two nucleotides on the 5′ end and the last two nucleotides on the 3′ end. The cap structure may increase resistance of the antisense oligonucleotide to exonucleases without compromising molecular interactions with the target sequence or cellular machinery. Such modifications may be selected on the basis of their increased potency in vitro or in vivo. The cap can be present at the 5′-terminus (5′-cap) or at the 3′-terminus (3′-cap) or can be present on both ends. In certain aspects, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In certain aspects, the antisense oligonucleotide forms a secondary structure at 18° C., but does not form a secondary structure at about 37° C. In some aspects, the antisense oligonucleotide does not form a secondary structure at about 18° C. or at about 37° C. In some aspects, the antisense oligonucleotide does not form a secondary structure at any temperature. In some aspects, the antisense oligonucleotide does not form a secondary structure at 37° C. In particular aspects, the secondary structure is a hairpin loop structure.

In certain aspects, the antisense oligonucleotide is chemically synthesized. In certain aspects, the antisense oligonucleotide is manufactured by solid phase organic synthesis. In some aspects, the synthesis of the antisense oligonucleotide is carried out in a synthesizer equipped with a closed chemical column reactor using flow-through technology. In some aspects, each synthesis cycle sequence on the solid support consists of multiple steps, which are carried out sequentially until the full-length antisense oligonucleotide is obtained. In certain aspects, the antisense oligonucleotide may be incorporated in a liposome formulation for systemic delivery. Suitable formulations are disclosed in U.S. Pat. No. 9,744,187, which is incorporated by reference herein in its entirety.

In certain aspects, the antisense oligonucleotide is stored in a liquid form. In some aspects, the antisense oligonucleotide is lyophilized prior to storing. In some aspects, the lyophilized antisense oligonucleotide is dissolved in water prior to use. In other aspects, the lyophilized antisense oligonucleotide is dissolved in an organic solvent prior to use.

In certain aspects, the antisense oligonucleotide is incubated with the tumor cells in a chamber. In certain aspects, the antisense oligonucleotide is incubated with the tumor cells outside of a chamber.

### Tumor Cells

In some aspects, the tumor cells are cancer cells selected from the group consisting of glioma cells, astrocytoma cells, hepatocarcinoma cells, breast cancer cells, head and neck squamous cell cancer cells, lung cancer cells, liver cancer cells, renal cell carcinoma cells, hepatocellular carcinoma cells, gall bladder cancer cells, classical Hodgkin's lymphoma cells, esophageal cancer cells, uterine cancer cells, rectal cancer cells, thyroid cancer cells, melanoma cells, colorectal cancer cells, prostate cancer cells, ovarian cancer cells, bone cancer cells, smooth muscle cells and pancreatic cancer cells.

In some aspects, the tumor cells are glioma cells. In some aspects, the tumor cells are recurrent malignant glioma cells. In some aspects, the tumor cells are glioblastoma cells. In some aspects, the tumor cells are astrocytoma cells. In some aspects, the tumor cells are astrocytoma cells, wherein astrocytoma is the grade II astrocytoma, AIII (IDH1 R132H mutant grade III astrocytoma), AIII-G (IDH1 wild-type grade III with characteristics of glioblastoma multiforme astrocytoma), or grade IV astrocytoma (glioblastoma multiforme). Most gliomas, particularly grades II through IV are astrocytomas, and thus, the terms glioma and astrocytoma are used interchangeably.

In some aspects, the tumor cells are derived from a subject. Accordingly, the neoantigens and compositions disclosed herein may be used to treat and immunize the subject against various cancers.

In some aspects, the tumor cells express a growth factor or a growth factor receptor. In some aspects, the tumor cells express the insulin-like growth factor 1 receptor (IGF-1R).

In some aspects, the tumor cells induced to produce neoantigens may be obtained directly by surgical excision of the tumor. In some aspects, the tumor cells may have been grown in vitro as primary cell lines derived from surgically excised tumor. In some aspects, the tumor cells are derived from one or more primary human glioblastoma cell lines. In some aspects, the tumor cells are primary human glioblastoma cells.

In some aspects, the tumor cells are removed from the patient using a tissue morselator. The extraction device preferably combines a high-speed reciprocating inner cannula within a stationary outer cannula and electronically controlled variable suction. The outer cannula has a diameter in the range of about 1 mm-4 mm. For example, the diameter may be 1.1 mm, 1.9 mm, 2.5 mm, or 3.0 mm. The outer cannula has a length in the range of about 5 cm to about 30 cm. For example, the length may be about 10 cm, 13 cm, or 25 cm. The instrument also relies on a side-mouth cutting and aspiration aperture located 0.6 mm from the blunt desiccator end. The combination of gentle forward pressure of the aperture into the tissue to be removed and suction draws the desired tissue into the side aperture, allowing for controlled and precise tissue resection through the reciprocal cutting action of the inner cannula. A key feature is the absence of a rotation blade; this avoids drawing unintended tissue into the aperture. An example of a suitable device is the Myriad® tissue aspirator (NICO Corporation® Indianapolis, Ind.), a minimally invasive surgical system which may be used for the removal of soft tissues with direct, microscopic, or endoscopic visualization. The shaved tissue is suctioned, gathered in to a collection chamber, and is collected in a sterile tissue trap. During collection of the tissue in the sterile tissue trap, blood is removed from the preparation.

Preferably, the morselator generates no heat at the resection site or along its shaft, and requires no ultrasonic energy for tissue removal. Thus, in particular aspects, the tumor tissue is morselized tumor tissue (i.e. tumor shaved tissue obtained by side-mouth cutting in the absence of heat, and optionally in the absence of ultrasonic treatment). Advantageously, the aspirator-extract and morselized tissue has higher viability than tissue removed by other methods. It is believed that the extraction process maintains higher tumor cell viability in part due to restricting exposure of the tumor cells to high temperatures during removal. For example, the methods herein do not expose tumor cells to above 25° C. during removal. In some aspects, the cells are not exposed to temperatures above body temperature, i.e., about 37° C.

The amount of tumor tissue obtained from the subject may vary. In some aspects, at least 1 gram, at least 5 grams, at least 10 grams or at least 20 grams of wet tumor tissue is obtained from the patient. In some aspects, an amount in the range of about 1 grams to 20 grams of wet tumor tissue may be obtained from the patient. The tissue is removed from the sterile tissue trap and disaggregated by pipetting with a sterile pipette to break up large tissue fragments. The disaggregated cell suspension is then placed onto sterile tissue culture plates in serum-containing media, and incubated in a tissue culture incubator. This plating step serves to enrich the desired cells by adherence, and also helps to remove debris from the preparation. After a predetermined incubation time, the cells are removed from the plates. The pre-incubation time may be in the range of about 1 hour-24 hours. For example, the pre-incubation time may be about 1 hour, about 2 hours, about 4 hours, about 6 hours, about 12 hours, or about 24 hours. The cells may be removed by scraping, by chemical methods (e.g. EDTA) or by enzymatic treatment (e.g. trypsin).

In some aspects, the tumor cells are sorted before being used for preparation of neoantigens. In some aspects, the cells are enriched by selecting for one or more cellular markers before being used for preparation of neoantigens. The selection may be performed, for example, using beads or by cell sorting techniques known to those of skill in the art. In some aspects, the cells used for preparation of neoantigens are enriched for one or more markers.

Optionally, the tumor cells used in the preparation of neoantigens may be enriched for certain cell types. Nestin, a cytoskeleton-associated class VI intermediate filament (IF) protein, has traditionally been noted for its importance as a neural stem cell marker. The inventors have discovered that in certain brain tumor samples, cells positive for nestin (nestin+cells) are enriched compared to benign tissue. In some aspects, a subject's tumor can be biopsied to assess the degree of nestin expression, and therefore, in certain aspects, the cells used for preparation of neoantigens are enriched for Nestin+cells compared to benign tissue. Without being bound by theory, it is thought that nestin provides a marker associated with antigens suitable useful in producing an anti-tumor immune response. Accordingly, the cells used for the preparation of neoantigens may be enriched for nestin+cells compared to the tumor cell population as a whole.

### Containers

In some aspects, the tumor cells are contained in a physical container or vessel where production of neoantigens takes place. Suitable containers include a chamber, such as a diffusion or biodiffusion chamber having pores or a membrane that contains pores. In some aspects, the tumor cells are contained in a vessel with a single membrane suspended in an outer vessel, such as, for example, Millipore culture inserts in 24 well plates. In some aspects, the tumor cells are contained in a chamber, diffusion chamber or a biodiffusion chamber, which is placed in a cell culture plate. In some aspects, the cell culture plate contains phosphate buffered saline (PBS).

In certain aspects, the pores, in a container allow passage of small molecules but not passage of cells (i.e., the cells cannot leave or enter the chamber). In some aspects, the diameter of the pores of the membrane allows passage of nucleic acids and other chemicals (such as, for example, cytokines produced by cells) through the pores. In some aspects, the diameter of the pores prevents passage of materials that are greater than 100 μm³ in volume into and out of the chamber. In some aspects, the diameter of the pores is such that it prevents the passage of neoantigens (produced from the tumor cells encapsulated within the chamber) through the pores.

In some aspects, the pores of the membrane have a diameter of about 0.25 μm or smaller. In some aspects, the pores range in diameter from 0.1 μm to 0.25 μm. For example, the pores may have a diameter of about 0.1 μm. See also, Lange, et al., J. Immunol., 1994, 153, 205-211 and Lanza, et al., Transplantation, 1994, 57, 1371-1375, each of which is incorporated herein by reference in their entireties. In certain aspects, diffusion chambers are constructed from 14 mm Lucite rings with 0.1 μm pore-sized hydrophilic Durapore membranes (Millipore, Bedford, Mass.).

In some aspects, the concentration of the antisense oligonucleotide that is brought to be in contact with the tumor cells in the biodiffusion chamber is in the range of about 1 μg/ml to 2 mg/ml, including all values and subranges that lie therebetween. In some aspects, the concentration of the antisense oligonucleotide is in the range of about 10 μg/ml to about 2 mg/ml. For example, in some aspects, the concentration of the antisense oligonucleotide is about 5 μg/ml, about 10 μg/ml, about 50 μg/ml, about 100 μg/ml, about 500 μg/ml, about 1 mg/ml, about 1.5 mg/ml or about 2 mg/ml.

In some aspects, the volume of the chamber is 200 μL. In some aspects, the amount of antisense oligonucleotide that is brought to be in contact with the tumor cells in the biodiffusion chamber is in the range of about 1 μg to about 5 mg, for example, about 5 μg, about 10 μg, about 25 μg, about 40 μg, about 50 μg, about 100 μg, about 200 μg, about 300 μg, about 400 μg, about 500 μg, about 600 μg, about 700 μg, about 800 μg, about 900 μg, about 1 mg, about 1.5 mg, about 2 mg, about 2.5 mg, about 3 mg, about 3.5 mg, about 4 mg, about 4.5 mg, or about 5 mg, including all values and subranges that lie therebetween.

Tumor cells can be placed in a diffusion chamber in varying numbers. In certain aspects, about 1×10⁴ to about 5×10⁶ tumor cells are placed in each diffusion chamber. In some aspects, about 1×10⁵ to about 1.5×10⁶ tumor cells are placed in the diffusion chamber. In some aspects, about 5×10⁵ to 1×10⁶ tumor cells are placed in the chamber. In some aspects, about 10⁶ tumor cells are placed in the chamber. In particular aspects, the volume of the chamber used to produce neoantigens is about 200 μl and contains 5 μg/ml to 2 mg/ml antisense oligonucleotide and about 50,000 to 1,000,000 cells.

In certain aspects, it may be preferable to maintain the ratio of cells to antisense oligonucleotide in a chamber. In certain aspects a chamber may contain about 2 μg of antisense oligonucleotide and between 750,000 and 1,250,000 cells; for example 1,000,000 cells. The ratio of cells to antisense oligonucleotide may thus be in a range from about 3.75×10⁵ to about 6.25×10⁵ per μg antisense oligonucleotide; for example, about 5.0×10⁵ cells per μg. In some aspects, a chamber may contain about 40 μg of antisense oligonucleotide and between 750,000 and 1,250,000 cells; for example 1,000,000 cells. The ratio of cells to antisense oligonucleotide may thus be in a range from about 1.87×10⁴ to about 3×10⁴ per μg antisense oligonucleotide; for example, about 2.5×10⁴ cells per μg. In some aspects, a chamber may contain about 400 μg of antisense oligonucleotide and between 750,000 and 1,250,000 cells; for example 1,000,000 cells. The ratio of cells to antisense oligonucleotide may thus be in a range from about 1.87×10³ to about 3×10³ per μg antisense oligonucleotide; for example, about 2.5×10³ cells per μg.

In some embodiments, the tumor cells may be contacted with a first antisense oligonucleotide before the tumor cells are contacted with a second antisense oligonucleotide in the biodiffusion chamber, i.e., in some embodiments, the tumor cells may be “pre-treated” with a first antisense oligonucleotide. In some aspects, the first antisense oligonucleotide and the second oligonucleotide are the same. In some aspects, the first antisense oligonucleotide and the second oligonucleotide are different.

In some embodiments, the tumor cells are pre-treated with the first antisense oligonucleotide for a time period in the range of about 5 minutes to about 48 hours, for example, about 15 min, about 30 min, about 45 min, about 1 hour, about 2 hours, about 3 hours, about 4 hours, about 5 hours, about 10 hours, about 12 hours, about 18 hours, or about 24 hours, including all values and subranges that lie therebetween. In some embodiments, the tumor cells are pre-treated with the first antisense oligonucleotide at a temperature in the range of about 10° C. to about 40° C., for example, about 15° C., about 18° C., about 20° C., about 22° C., about 24° C., about 26° C., about 28° C., about 30° C., about 32° C., about 34° C., about 36° C., about 37° C., about 38° C., about 39° C. or about 40° C., including all values and subranges that lie therebetween.

In some embodiments, the tumor cells may be pre-treated with a first antisense oligonucleotide overnight before the tumor cells are contacted with a second antisense oligonucleotide in the biodiffusion chamber for a period of time. The period of time is not limited, and may be in a range of about 5 minutes to about 48 hours, for example, about 15 min, about 30 min, about 45 min, about 1 hour, about 2 hours, about 3 hours, about 4 hours, about 5 hours, about 10 hours, about 12 hours, about 18 hours, or about 24 hours, including all values and subranges that lie therebetween. In some aspects, after the period of time, the chamber is irradiated. In some aspects, the first antisense oligonucleotide and the second oligonucleotide are the same. In some aspects, the first antisense oligonucleotide and the second oligonucleotide are different.

In some aspects, the tumor cells are pre-treated with the first antisense oligonucleotide which is present in an amount of about 1 μg to about 5 mg, for example, about 5 μg, about 10 μg, about 25 μg, about 40 μg, about 50 μg, about 100 μg, about 200 μg, about 300 μg, about 400 μg, about 500 μg, about 600 μg, about 700 μg, about 800 μg, about 900 μg, about 1 mg, about 1.5 mg, about 2 mg, about 2.5 mg, about 3 mg, about 3.5 mg, about 4 mg, about 4.5 mg, or about 5 mg, including all values and subranges that lie therebetween.

In some aspects, the tumor cells are pre-treated with the first antisense oligonucleotide which is present in a concentration of about 1 μg/ml to about 50 mg/ml, for example, about 5 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1 mg/ml, about 1.5 mg/ml, about 2 mg/ml, about 10 mg/ml, about 20 mg/ml, about 30 mg/ml, about 40 mg/ml, or about 50 mg/ml, including all values and subranges that lie therebetween.

### Neoantigens and Immunogenic Compositions Comprising Neoantigens

The disclosure also provides neoantigens prepared by the methods disclosed herein. Further, the disclosure provides, immunogenic compositions comprising the neoantigens disclosed herein. In some aspects, the neoantigens may be uniquely produced by a subject's tumor cells. In some aspects, the neoantigens may be antigens that are produced by tumor cells derived from several different subjects.

In some aspects, neoantigen compositions disclosed herein do not contain intact tumor cells or viable tumor cells. Without being bound by theory, it is thought that in some aspects, the neoantigens produced by the methods disclosed herein are part of microvesicles.

In some aspects, the volume of the neoantigens does not permit passage of the neoantigens through the pores in the membrane of the diffusion chamber. In some aspects, the volume of the neoantigens is greater than about 100 μm³. In some aspects, the volume of the neoantigens permits passage of the neoantigens through the pores in the membrane of the diffusion chamber. In some aspects, the volume of the neoantigens is less than about 100 μm³. In some aspects, the volume of the neoantigens is about 100 μm³.

In some aspects, the immunogenic compositions disclosed herein further comprise at least one pharmaceutically acceptable adjuvant, excipient, buffer, diluent and the like. For example, the immunogenic compositions may contain sodium phosphate, sodium chloride, and/or histidine. Sodium phosphate may be present at about 10 mM to about 50 mM, about 15 mM to about 25 mM, or about 25 mM; in particular cases, about 22 mM sodium phosphate may be present. Histidine may be present at about 0.1% (w/v) to about 2.5% (w/v) or about 0.7% (w/v) to about 1.5% (w/v). In some aspects, histidine may be present at about 0.1% (w/v), about 0.5% (w/v), about 0.7% (w/v), about 1% (w/v), about 1.5% (w/v), about 2% (w/v), or about 2.5% (w/v). Sodium chloride, when present, may be at about 50 mM to about 250 mM, preferably about 100 mM to about 200 mM. In some aspects, sodium chloride is present at about 150 mM.

In some aspects, the pharmaceutically acceptable excipient may comprise dextrose, water, glycerol, sterile isotonic aqueous buffer, and combinations thereof. In some aspects, the pharmaceutically acceptable excipient may comprise phosphate buffered saline, sterile saline, lactose, sucrose, calcium phosphate, dextran, agar, pectin, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some aspects, the compositions disclosed herein further comprise minor amounts of emulsifying agents, or pH buffering agents.

In some aspects, the immunogenic compositions disclosed herein may further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some aspects, the immunogenic compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

The immunogenicity of the compositions disclosed herein may be enhanced by the use of an effective amount of one or more adjuvants. Adjuvants have been used experimentally to promote a generalized increase in immunity against antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2nd Edition),” herein incorporated by reference in its entirety for all purposes, is envisioned within the scope of this disclosure.

Exemplary adjuvants include complete Freund's adjuvant, incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/Tween® 80 emulsion. In other preferred aspects, Alum such as 2% Alhydrogel (Al(OH)3) is used. In some aspects, the adjuvant may be a paucilamellar lipid vesicle; for example, Novasomes®. Novasomes® are paucilamellar nonphospholipid vesicles ranging from about 100 nm to about 500 nm. They comprise Brij 72, cholesterol, oleic acid and squalene. Novasomes have been shown to be an effective adjuvant (see, U.S. Pat. Nos. 5,629,021, 6,387,373, and 4,911,928. In particular aspects, the adjuvant is a saponin Fraction A matrix, a saponin Fraction C matrix or a combination of both.

In some aspects, the composition is in a lyophilized powder suitable for reconstitution, a liquid solution, suspension, emulsion, tablet, pill, capsule, sustained release formulation, or powder. In some aspects, delivery vehicles such as liposomes, nanocapsules, microparticles, microspheres, lipid particles, vesicles, and the like, may be used for administering the compositions disclosed here to the subject.

### Immunization and Treatment Methods

The disclosure provides methods of inducing an immune response in a subject, comprising administering to the subject a therapeutically effective amount of the neoantigens and/or the immunogenic compositions disclosed herein. In some aspects, the induction of the immune response immunizes the subject against the development of cancer. In some aspects, a primary immune response is induced, while in other aspects, a secondary immune response is induced. The disclosure also provides methods of inducing resistance to growth of a cancer in a subject, comprising administering to the subject a therapeutically effective amount of the neoantigens and/or the immunogenic compositions disclosed herein.

Further, the disclosure provides methods of inducing regression of a cancer in a subject, comprising administering to the subject a therapeutically effective amount of the neoantigens and/or the immunogenic compositions disclosed herein. The disclosure also provides methods of treating a cancer in a subject, comprising administering to the subject a therapeutically effective amount of the neoantigens and/or the immunogenic compositions disclosed herein.

The disclosure also provides methods of inducing an immune response in a first subject, comprising administering to the first subject a therapeutically effective amount of the neoantigens and/or the immunogenic compositions disclosed herein; methods of inducing resistance to growth of a cancer in a first subject, comprising administering to the first subject a therapeutically effective amount of the neoantigens and/or the immunogenic compositions disclosed herein; methods of inducing regression of a cancer in a first subject, comprising administering to the first subject a therapeutically effective amount of the neoantigens and/or the immunogenic compositions disclosed herein; and methods of treating a cancer in a subject, comprising administering to the subject a therapeutically effective amount of the neoantigens and/or the immunogenic compositions disclosed herein.

In some aspects, the neoantigens are produced using tumor cells derived from a second subject in any one of the methods of preparing neoantigens disclosed herein. In some aspects, the first subject and the second subject are the same. In some aspects, the first subject and the second subject are different.

In some aspects, administration of the neoantigen compositions disclosed herein for a therapeutically effective time reduces or eliminates return of the cancer in the subject. In certain aspects, the methods disclosed herein result in a reduction of tumor volume associated with the cancer in the subject. In some aspects, the methods disclosed herein induces elimination of the tumor in the subject. In some aspects, the methods disclosed herein inhibit regrowth of the tumor for at least 3 months, at least 6 months, at least 12 months, or at least 36 months. In some aspects, the methods disclosed herein delay the onset of tumor growth and the symptoms associated with tumor growth.

In some aspects, neoantigens having a volume more than the 100 μm³ and neoantigens having a volume less than 100 μm³ may be administered concurrently. In some aspects, neoantigens having a volume more than the 100 μm³ and neoantigens having a volume less than 100 μm³ may be administered sequentially. Neoantigens having a volume more than the 100 μm³ or neoantigens having a volume less than 100 μm³ may be administered first, followed by the neoantigens of greater or smaller volume.

In some aspects, the subject may be administered a dose of antisense oligonucleotide separately from administering the neoantigen compositions disclosed herein. In some aspects, the antisense oligonucleotide may be administered in free form or as a liposome. In some aspects, the nucleic acid backbone of the antisense oligonucleotide comprises at least one p-ethoxy backbone linkage.

In some aspects, the methods disclosed herein may be combined with other therapies; for example, radiation therapy. In certain aspects, the radiation therapy includes, but is not limited to, internal source radiation therapy, external beam radiation therapy, and systemic radioisotope radiation therapy. In certain aspects, the radiation therapy is external beam radiation therapy. In some aspects, the external beam radiation therapy includes, but is not limited to, gamma radiation therapy, X-ray therapy, intensity modulated radiation therapy (IMRT), and image-guided radiation therapy (IGRT). In certain aspects, the external beam radiation therapy is gamma radiation therapy. Radiation may be administered before, during or after administration of the neoantigens and compositions disclosed herein.

In some aspects, the methods described herein may be used in the same subject, alone or in combination with radiation or chemotherapy. In some aspects, the chemotherapeutic drug is temozolomide. In some aspects, the methods disclosed herein are preferably used as a first-line therapy. Without being bound by theory, it might be desirable to use the methods disclosed herein as a first-line therapy because the subject's immune system can be inhibited by other therapies, reducing the therapeutic benefit of the methods disclosed herein.

In some aspects, the subject may have been newly diagnosed with cancer. In some aspects, the subject may have been diagnosed with a cancer that has recurred after being previously treated with standard-of-care therapies. In some aspects, the subject is one who has not been previously treated with any therapeutic approaches that are immunosuppressive. In particular aspects, eligible subjects are over 18 years of age and have a Karnofsky score of 60 or above. In some aspects, the subjects do not have bihemispheric disease and/or do not have an autoimmune disease.

### Dosage and Administration

In some aspects, the neoantigens and/or the immunogenic compositions comprising neoantigens disclosed herein are not administered in a device. In some aspects, the neoantigens and/or the immunogenic compositions comprising neoantigens disclosed herein are not administered in a biodiffusion chamber.

In some aspects, the neoantigens and/or the immunogenic compositions comprising neoantigens disclosed herein may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. Typically, the systemic, parenteral administration is intramuscular injection. As used herein, the term “mucosal administration” includes oral, intranasal, intravaginal, intra-rectal, intra-tracheal, intestinal and ophthalmic administration. In some aspects, administration is intramuscular.

In certain aspects, the neoantigens and/or the immunogenic compositions comprising neoantigens disclosed herein are administered pre-operatively; for example prior to surgery to reduce tumor burden. For example, the neoantigens and/or the immunogenic compositions may be administered up to 24 hours, up to 36 hours, up to 48 hours or up to 72 hours before surgery. In particular aspects, the immunogenic composition may be administered about 48 hours to about 72 hours before surgery. Typically, in such circumstances, the administration is by intravenous bolus. In certain aspects, the neoantigens and/or the immunogenic compositions comprising neoantigens disclosed herein are administered post-operatively. For example, the neoantigens and/or the immunogenic compositions may be administered up to 24 hours, up to 36 hours, up to 48 hours or up to 72 hours after surgery. In particular aspects, the immunogenic composition may be administered about 48 hours to about 72 hours after surgery.

The neoantigens and/or the immunogenic compositions comprising neoantigens disclosed herein may be administered on a single dose schedule or a multiple dose schedule. Multiple doses may be used in a primary immunization schedule or in a booster immunization schedule. In a multiple dose schedule, the various doses may be given by the same or different routes e.g., a parenteral prime and mucosal boost, a mucosal prime and parenteral boost, etc. In some aspects, a follow-on boost dose is administered about 2 weeks, about 3 weeks, about 4 weeks, about 5 weeks, or about 6 weeks after the prior dose. In other aspects, the compositions disclosed herein are administered only once, yet they induce an anti-tumor response.

In some aspects, a follow-on boost dose of immunogenic compositions comprising neoantigens disclosed herein may be administered to a subject. Such booster approaches may be used subsequent to administering free neoantigens. In other aspects, however, the booster approach may be used when the subject has already been implanted with one or more biodiffusion chambers comprising irradiated tumor cells from the subject's tumor and an antisense oligonucleotide directed to insulin-like growth factor receptor-1. Such biodiffusion chambers and methods of use thereof are described in detail in U.S. application Ser. No. 15/095,877 and WO 2018165528A1, both of which are incorporated by reference herein in their entireties.

In some aspects, the compositions are administered to a subject in need thereof at a therapeutically effective dose. The dosage of the compositions disclosed herein will depend on factors such as the type of cancer being treated, the age, weight and health of the subject, and route of administration.

The dose, as measured in μg, may be the total weight of the neoantigens as measured using protein concentration assay, such as either A280 or ELISA. A therapeutically effective dosage of the neoantigens and/or the immunogenic compositions comprising neoantigens disclosed herein, including for pediatric administration, may be in the range of about 30 μg to about 500 μg, about 100 μg to about 500 μg, about 150 μg to about 500 μg, about 200 μg to about 500 μg or about 300 μg to about 500 μg. In particular aspects, the dose is about 120 μg, administered with alum. In some aspects, a pediatric dose may be in the range of about 30 μg to about 90 μg.

In some aspects, the dose may be administered in a volume of about 0.1 mL to about 1.5 mL, about 0.3 mL to about 1.0 mL, about 0.4 mL to about 0.6 mL, or about 0.5 mL, which is a typical amount.

In particular aspects, the dose may comprise a neoantigen protein concentration of about 175 μg/mL to about 325 μg/mL, about 200 μg/mL to about 300 μg/mL, about 220 μg/mL to about 280 μg/mL, or about 240 μg/mL to about 260 μg/mL.

All patents, patent applications, references, and journal articles cited in this disclosure are expressly incorporated herein by reference in their entireties for all purposes. The materials, methods, and examples are illustrative only and are not intended to be limiting.

### EMBODIMENTS

Embodiment 1: A method of producing neoantigens, comprising:

(a) contacting tumor cells from a first subject with a first antisense oligonucleotide to produce neoantigens, wherein the culture is irradiated before or after step (a) and wherein the first antisense oligonucleotide is an insulin-like growth factor 1 receptor (IGF-1R) antisense oligodeoxynucleotide; and

(b) recovering the neoantigens.

Embodiment 1.1: The method of embodiment 1, wherein recovering the neoantigens comprises separating the neoantigens from the tumor cells using a pore and/or centrifugation.

Embodiment 2. The method of embodiment 1, comprising contacting tumor cells with a second antisense oligonucleotide before step (a).

Embodiment 3. The method of embodiment 2, wherein the second antisense oligonucleotide is the same as the first antisense oligonucleotide.

Embodiment 4. The method of any one of embodiments 2 and 3, wherein tumor cells are contacted with an amount of the second antisense oligonucleotide which is greater than the amount of the first antisense oligonucleotide.

Embodiment 5. The method of any one of embodiments 1-4, wherein the tumor cells are selected from the group consisting of glioma cells, glioblastoma cells, astrocytoma cells, hepatocarcinoma cells, breast cancer cells, head and neck squamous cell cancer cells, lung cancer cells, liver cancer cells, renal cell carcinoma cells, hepatocellular carcinoma cells, gall bladder cancer cells, classical Hodgkin's lymphoma cells, esophageal cancer cells, uterine cancer cells, rectal cancer cells, thyroid cancer cells, melanoma cells, colorectal cancer cells, prostate cancer cells, ovarian cancer cells, bone cancer cells, smooth muscle cells and pancreatic cancer cells.

Embodiment 6. The method of any one of embodiments 1-5, wherein the tumor cells express the insulin-like growth factor 1 receptor (IGF-1R).

Embodiment 7. The method of any one of embodiments 1-6, wherein the tumor cells are glioblastoma cells.

Embodiment 8. The method of any one of embodiments 1-7, wherein the culture is in a biodiffusion chamber.

Embodiment 9. The method of any one of embodiments 1-8, wherein the culture is in a ring-shaped biodiffusion chamber.

Embodiment 10. The method of any one of embodiments 8 and 9, wherein the biodiffusion chamber comprises a cell-impermeable membrane.

Embodiment 11. The method of embodiment 10, wherein the membrane comprises pores with a diameter in the range of about 0.25 μm or smaller.

Embodiment 12. The method of any one of embodiments 10 and 11, wherein the membrane comprises pores with a diameter in the range of about 0.1 μm.

Embodiment 13. The method of any one of embodiments 1-12, wherein the first antisense oligonucleotide comprises the amino acid sequence of SEQ ID NO: 2 and wherein the nucleic acid backbone of the first antisense oligonucleotide comprises at least one phosphorothioate linkage.

Embodiment 14. The method of embodiment 13, wherein the nucleic acid backbone of the first antisense oligonucleotide comprises at least one p-ethoxy backbone linkage.

Embodiment 15. The method of any one of embodiments 1-14, wherein the culture is irradiated with about 5Gy to about 100Gy radiation.

Embodiment 16. An immunogenic composition comprising the neoantigens produced by the method of any one of embodiments 1-15.

Embodiment 17. An immunogenic composition comprising the neoantigens produced by the method of any one of embodiments 11 and 12.

Embodiment 18. The immunogenic composition of embodiment 17, wherein the volume of the neoantigens does not permit passage of the neoantigens through the pores.

Embodiment 19. The immunogenic composition of embodiment 18, wherein the neoantigens are greater than 100 μm³ in volume.

Embodiment 20. A method of inducing an anti-tumor immune response in a second subject in need thereof, comprising administering to the second subject a therapeutically effective amount of the neoantigens produced by the method of any one of embodiment 1-15 or the immunogenic composition of any one of embodiment 16-19.

Embodiment 21. The method of embodiment 20, wherein the induction of the immune response immunizes the second subject against the development of a cancer.

Embodiment 22. A method of inducing resistance to growth of a cancer in a second subject in need thereof, comprising administering to the second subject a therapeutically effective amount of the neoantigens produced by the method of any one of embodiments 1-15 or the immunogenic composition of any one of embodiments 16-19.

Embodiment 23. A method of inducing regression of a cancer in a second subject in need thereof, comprising administering to the second subject a therapeutically effective amount of the neoantigens produced by the method of any one of embodiments 1-15 or the immunogenic composition of any one of embodiments 16-19.

Embodiment 24. A method treating a cancer in a second subject in need thereof, comprising administering to the second subject a therapeutically effective amount of the neoantigens produced by the method of any one of embodiments 1-15 or the immunogenic composition of any one of embodiments 16-19.

Embodiment 25. The method of any one of embodiments 21-24, wherein the cancer is selected from the group consisting of glioma, glioblastoma, astrocytoma, hepatocarcinoma, breast cancer, head and neck squamous cell cancer, lung cancer, liver cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, bone cancer, smooth muscle and pancreatic cancer.

Embodiment 26. The method of any one of embodiments 21-25, wherein the cancer is a glioblastoma.

Embodiment 27. The method of any one of embodiments 20-26, wherein the neoantigens produced by the method of any one of embodiments 1-15 or the immunogenic composition of any one of embodiments 16-19 are not administered in a device.

Embodiment 28. The method of embodiment 27, wherein the device is a biodiffusion chamber.

Embodiment 29. The method of any one of embodiments 20-26, wherein the neoantigens produced by the method of any one of embodiments 1-15 or the immunogenic composition of any one of embodiments 16-19 are administered to the second subject systemically.

Embodiment 30. The methods of embodiments 20-29, comprising administering to the second subject a follow-on boost dose of the neoantigens produced by the method of any one of embodiments 1-15 or the immunogenic composition of any one of embodiments 16-19.

Embodiment 31. The method of any one of embodiments 20-30, wherein the first subject and the second subject are the same.

Embodiment 32. The method of any one of embodiments 20-30, wherein the first subject and the second subject are different.

### EXAMPLES

### Example 1

Production of Neoantigens from Glioma Cells and Detection Thereof

In order to produce tumor antigens or neoantigens, biodiffusion chambers were prepared with 10⁶ GL261 cells in 200 μl phosphate-buffered saline (PBS) alone, or with 400 μg, 40 μg, or 2 μg of NOBEL having a fully phosphorothioate backbone, irradiated at 5 Gy and then placed in 6 well culture plates containing 5 ml PBS. The contents of the biodiffusion chamber are referred to as the “chamber contents,” while the contents of the cell culture plate (where the chamber was incubated in PBS) are referred to herein as the “supernatant.” After incubation for approximately 18 hours at 37° C., chamber contents containing putative tumor antigens were recovered using a pipette to pierce the membrane and withdraw the contents. Without being bound by theory, it is thought that much of the cell debris got stuck to the chamber membranes, while small molecules diffused into the PBS in the cell culture plate that the chambers were incubated in. The recovered chamber contents containing putative tumor antigens included cell products and cell constituents, soluble and in suspension, but no intact cells. The putative tumor antigens may be associated with microvesicle particles.

CD4 T cells immunized against GL261 glioma specific antigens were obtained from immunized mice. C57BL/6 mice were immunized against GL261 glioma-specific antigens by one of following methods: (1) implantation of viable GL261 cells in the flank (50% become immune); (2) inoculation of a mix of GL261 cells and the phosphorothioate-linked antisense oligonucleotide NOBEL into the flank; or (3) implantation of a diffusion chamber containing irradiated GL261 cells and NOBEL into the flank, as specified below in each experiment. Immunized mice prevent the growth of 10⁵ GL261 cells stereotactically implanted into their cerebral cortex. This therapeutic immune response is dependent upon the presence of CD4 T cells that make IFNγ. These cells were recovered from the spleens and lymph nodes of immunized mice and used to detect the tumor antigens that they are specific for in vitro.

The chamber contents were incubated with Dendritic cells (DC) derived from the bone marrow of non-immune C57BL/6 mice in 10 ml RPMI1640 10% FBS for an additional 18 hours. These “pulsed” DC cells were used to present putative neoantigens to T cells from immunized mice in the following manner. The “pulsed” DC were recovered, spun down in a centrifuge at 1200 RPM for 7 minutes, washed by a second centrifugation in prewarmed PBS, resuspended in RPM11640 10% FBS at 50,000 cells per 100 μl and incubated with 10⁵ primed CD4 T cells from GL261-immune C57BL/6 mice for 18 hours in ELISPOT plates coated with IFNγ-specific antibodies.

The production of IFNγ by CD4 T cells detected in an IFNγ ELISPOT assay was used to quantify the amount of neoantigens present. The number of responding T cells was correlated with the amount and immunogenicity of neoantigens present. The plates were then washed and a second IFNγ-specific antibody conjugated to streptavidin, biotin-HRP and AEC chromogenic substrate used to detect the spots where T cells had produced IFNγ. To control for possible non-specific effects of NOBEL, DC without antigen exposure were added with 400 μg, 40 μg, or 2 μg of NOBEL to primed T cells with no response noted.

Substantial numbers of T cells producing IFNγ were only seen with the contents of chambers in which irradiated GL261 cells and NOBEL had been incubated. Higher responses were seen for those with 40 μg and 400 μg NOBEL in the absence of pre-treatment. Pretreatment of GL261 cells with 4 mg NOBEL before addition to the chambers can be used to enhance neoantigen production when only a low amount of NOBEL (2 μg) is included in the chamber. These results are described in detail below.

### Example 2

Production of Neoantigens Using Irradiated Tumor Cells Incubated with NOBEL

Mice were injected with 10⁶ GL261 cells from ex-vivo tissues in the flank sub-cutaneously. Tumors were allowed to develop for 14 days and mice were treated intra-venously with 0.1 mg NOBEL. Mice were monitored for sixty days and then mice which both did, and did not develop tumors were sacrificed and splenic CD4+ T cells were isolated.

Naïve dendritic cells (DC) were isolated from bone marrow of bl/6 mice and matured for at least one week using GM-CSF treatment. DCs were pulsed overnight with various antigen formulations (chamber contents from the diffusion chambers described below), collected, and washed. These pulsed DCs were then incubated overnight with isolated immunized CD4+ T cells in a culture plate coated with IFN-gamma capture antibody. After processing, the IFN-gamma spots/well were counted as a measure of the ability of the chamber contents to activate immunized T cells.

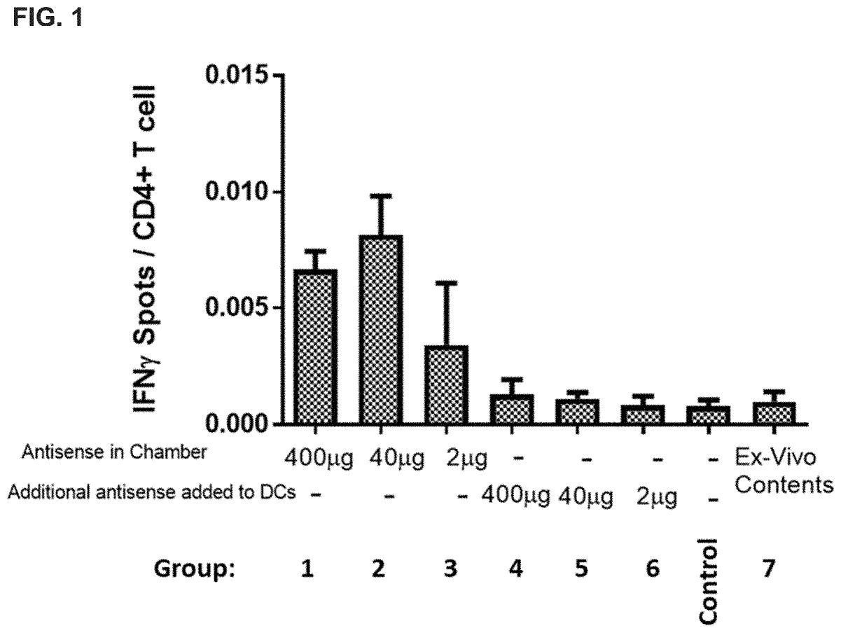

Seven different diffusion chambers were prepared as described below in Groups 1-7, and represented in FIG. 1. In chambers (1) to (3), the GL261 cells were pre-treated overnight with 4 mg NOBEL/1 million cells, before being incubated with 400 μg, 40 μg or 2 μg of NOBEL in the chambers. After pre-treatment, cells were harvested, washed, and resuspended in PBS. In chambers (4) to (7), the GL261 cells were not pre-treated with NOBEL. Also, as indicated below, while NOBEL was added to chambers (1) to (3), no NOBEL was added to chambers (4)-(7). When the contents of the irradiated chambers (4) to (6) were used to pulse DCs, then NOBEL was added at that stage, as indicated below.

(1) 1 million cells were pre-treated with 4 mg NOBEL and then, placed in a biodiffusion chamber, 400 μg NOBEL was added to the chamber. The chamber was irradiated at 5 Gy and incubated overnight
(2) 1 million cells were pre-treated with 4 mg NOBEL and then, placed in a biodiffusion chamber; 40 μg NOBEL was added to the chamber. The chamber was irradiated at 5 Gy and incubated overnight
(3) 1 million cells were pre-treated with 4 mg NOBEL and then, placed in a biodiffusion chamber; 2 μg NOBEL was added to the chamber. The chamber was irradiated at 5 Gy and incubated overnight
(4) 1 million cells were incubated in an irradiated chamber overnight and 400 μg NOBEL was added at DC pulse
(5) 1 million cells were incubated in an irradiated chamber overnight and 40 μg NOBEL was added at DC pulse
(6) 1 million cells were incubated in an irradiated chamber overnight and 2 μg NOBEL was added at DC pulse
(7) Contents from ex-vivo chamber (control)

Results: The ability of the contents of chambers (1) to (7) to activate immunized T-cells was evaluated by measuring IFN-gamma spots/CD4+ T-cell (see FIG. 1). The data showed that the addition of NOBEL in the chamber increased the ability of the chamber contents to activate immunized T-cells (Groups 1-3, above and in FIG. 1), as compared to control chamber contents (Group 7 in FIG. 1). Particularly, the contents of chambers in which the irradiated cells were incubated with 400 μg or 40 μg of NOBEL showed an increased ability to activate immunized T-cells as compared to contents of chambers in which the irradiated cells were incubated with 2 μg of NOBEL. Further, as shown in FIG. 1, the addition of the NOBEL to dendritic cells during the pulse, as was done in the case of contents of chambers Groups (4)-(6), did not have any significant effect on the ability of the contents of irradiated chambers Groups (4)-(6) to activate immunized T-cells.

### Example 3

### Effect of the Amount of NOBEL Used on the Production of Neoantigens

Mice were immunized with chambers containing 1 million GL261 cells that had been pre-treated overnight with 4 mg NOBEL/1 million cells and then incubated in chambers containing 2 μg NOBEL. Chambers were implanted in the flank for 48 hours. The immune response was allowed to develop for 35 days and then the mice were challenged intra-cranially with 100,000 GL261 cells. Survival was monitored for 60 days and CD4+ T cells were isolated from survivors for use in ELISPOT assay.

Four different diffusion chambers were prepared as described below Groups 1-4.

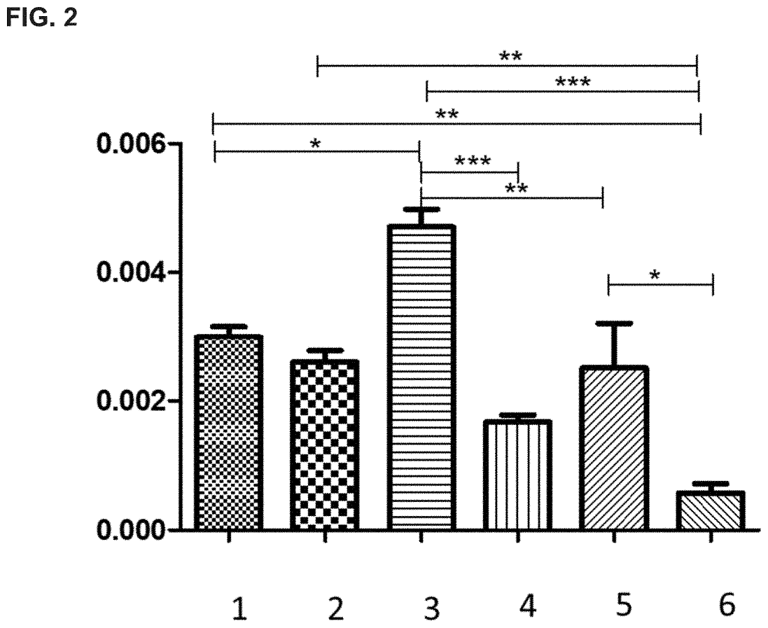

(1) 1 million cells were pre-treated overnight with 4 mg NOBEL and then placed in a biodiffusion chamber; 2 μg NOBEL was added to the chamber. The chamber was irradiated and incubated overnight in a cell culture plate containing PBS—Chamber contents as well as supernatant (contents of the cell culture plate where chamber was incubated in PBS) were recovered and tested (see FIG. 2, bar 1 (represents chamber contents) and bar 2 (represents supernatant))
(2) 1 million cells were placed in a chamber and 400 μg NOBEL was added to the chamber. The chamber was irradiated and incubated overnight in a cell culture plate containing PBS—Chamber contents as well as supernatant (from cell culture plate where chamber was incubated in PBS) were recovered and tested (see FIG. 2, bar 3 (represents chamber contents) and bar 4 (represents supernatant))
(3) 1 million GL261 cells were plated in cell culture well in 2 mL PBS and 4 mg antisense oligonucleotide added to the well supernatant—the supernatant was recovered and tested (see FIG. 2, bar 5)
(4) Ex-vivo chamber from mouse (see FIG. 2, bar 6)

In the case of (1) and (2) above, the contents of the chamber (referred to as the “chamber contents”) as well as the contents of the cell culture plate where the chamber was incubated in PBS (referred to as the “supernatant”) were recovered. The ability of the chamber contents and the supernatant to activate immunized T cells was measured, as described above in Example 1, and as shown in FIG. 2.

Results: The measurement of IFN-gamma spots/CD4+ T Cell showed that the contents of the chamber from (1) had a comparable ability to activate immunized T cells to the supernatant from (1). Both the chamber contents and supernatant of (1) had a measurably greater ability to activate immunized cells as compared to the contents of the control chamber (4) (see FIG. 2, bar 6). Moreover, the supernatant from (3) above also exhibited a similar ability to activate immunized T cells, as compared to chamber contents and the supernatant recovered from (1).

The contents of the irradiated chamber in which cells were treated with 400 μg of NOBEL (2) exhibited the highest ability of activate immunized T cells. The ability of the supernatant from (2) to activate immunized T cells was lower than the chamber contents of (2); and the chamber contents and the diffused contents of (1).

These results indicate that the amount of NOBEL used to treat the cells in the irradiated chamber overnight is positively correlated with the amount of neoantigens produced by the cells (as shown in FIG. 2, contents of irradiated chambers in which cells were treated with 400 μg of NOBEL had a greater ability to activate immunized cells, as compared to contents of irradiated chambers in which cells were treated with 2 μg of NOBEL). Further, the data also demonstrates that some antigens are less than 100 μm³ in size and the other antigens have a larger size. Thus enhanced immunization may be obtained using neoantigens purified by sizing and exclusion techniques, optionally by administering different sized antigens sequentially.

### Example 4

Effect of Pre-Treating Tumor Cells with NOBEL on the Production of Neoantigens

Mice were immunized with chambers containing 1 million GL261 cells that had been pre-treated overnight with 4 mg NOBEL/1 million cells and then incubated in the chambers containing 2 μg NOBEL. Chambers were implanted in the flank for 48 hours. The immune response was allowed to develop for 35 days and then the mice were challenged intra-cranially with 100,000 GL261 cells. Survival was monitored for 60 days and CD4+ T cells were isolated from survivors for use in ELISPOT assay.

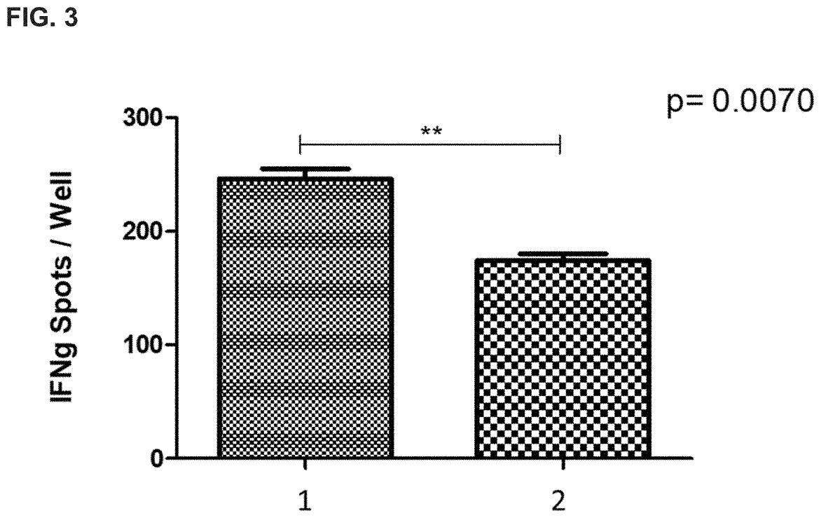

Two different chambers were prepared as described below, and as represented in FIG. 3:

(1) 1 million cells pre-treated with 4 mg NOBEL and then, placed in a chamber; 2 μg NOBEL was added to the chamber. The chamber was irradiated and incubated overnight. (FIG. 3, bar 1)
(2) 1 million cells were placed in a chamber; 2 μg NOBEL was added to the chamber. The chamber was irradiated and incubated overnight (FIG. 3, bar 2)

Results: The measurement of IFN-gamma spots/well showed that the contents of the chamber in which GL261 cells were pre-treated with NOBEL prior to incubation with 2 μg NOBEL in irradiated chamber overnight (see (1) above and in FIG. 3) had a greater ability to activate immunized T cells, as compared to the contents of the chamber in which the GL261 cells were not pre-treated with NOBEL (see (2) above and in FIG. 3). This experiment showed that the pre-treatment of tumor cells with NOBEL before culturing the cells in the biodiffusion chamber can be used to enhance the antigenicity of the chamber contents. Pre-treating with NOBEL might be advantageous, especially when only a low amount of NOBEL is used for incubation with tumor cells in the irradiated chamber.

### Example 5

Efficient Production of Glioma Antigens In Vitro Requires Treatment of Tumor Cells with NOBEL and Irradiation

Biodiffusion chambers containing 10⁶ GL261 cells were treated as shown below in Groups 1-5.

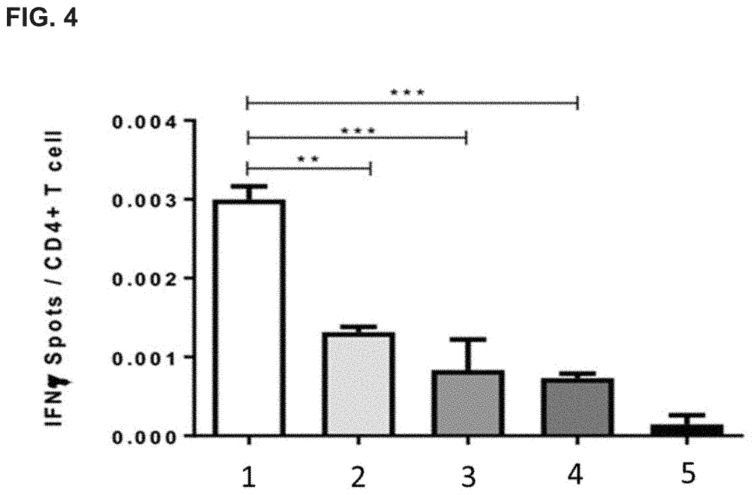

(1) 10⁶ GL261 cells were pre-treated with NOBEL in an amount of about 4 mg/10⁶ cells overnight. The cells were placed in a diffusion chamber, then NOBEL was added; and the chamber was irradiated at 5Gy. The chamber was placed in petri-plates containing PBS at 37° C. for 24 hours (FIG. 4, Bar 1).
(2) 10⁶ GL261 cells were pre-treated with NOBEL overnight. The cells were placed in a diffusion chamber, then NOBEL was added; the chamber was not irradiated. The chamber was placed in petri-plates containing PBS at 37° C. for 24 hours (FIG. 4, Bar 2).
(3) 10⁶ GL261 cells were placed in a diffusion chamber; the cells were not pre-treated. Then NOBEL was added to the chamber, and the chamber was irradiated at 5Gy. The chamber was placed in petri-plates containing PBS at 37° C. for 24 hours (FIG. 4, Bar 3).
(4) 10⁶ GL261 cells were pre-treated with NOBEL overnight. The cells were placed in a diffusion chamber. NOBEL was not added to the diffusion chamber. The chamber was irradiated at 5Gy. The chamber was placed in petri-plates containing PBS at 37° C. for 24 hours (FIG. 4, Bar 4).
(5) Background signal (FIG. 4, Bar 5)

Chamber contents were then incubated with bone marrow-derived DC and the DC incubated overnight with T cells from GL261-immune mice. T cells were then recovered and the number producing IFN-γ determined in ELISPOTs. Data is presented as spots per CD4 T cell.

Results: The contents from the chamber pre-treated with NOBEL and incubated with NOBEL in irradiated chambers had a significantly greater ability (by ANOVA, p<0.001) to activate immunized T-cells than the contents of any other chamber tested. See FIG. 4. These results show that each of the following steps—irradiation of the chamber, pre-treatment of cells with NOBEL, and incubation of the cells with NOBEL—contributes to producing neoantigens that can effectively activate immunized T-cells. Therefore, combining all the three steps results in an enhanced production of neoantigens.

### Example 6

Production of Neoantigens Using Human Glioblastoma (GBM) Cells Incubated with NOBEL

Neoantigens were prepared by treatment of a primary human GBM cell line in vitro, with or without 2 h pretreatment with 400 μg/ml NOBEL followed by placing of the tumor cells in 200 μL chambers with additional 4-400 μg NOBEL and irradiated with 100 Gy irradiation. The chamber contents were removed after 24 h incubation at 37° C. for pretreated cells and immediately for chambers containing cells that had not been pretreated.

The contents were used to pulse (overnight culture) dendritic cells prepared from the peripheral blood mononuclear cell (PBMC) of a glioblastoma (GBM) patient obtained before treatment. Further, T cells prepared from the same PBMC sample were added to the dendritic cells in an ELISPOT plate coated with antibodies to IFNγ for approximately 20 hrs. Spots identifying cells producing IFNγ were then developed using a second antibody to IFNγ, and conventional colorimetric approaches.

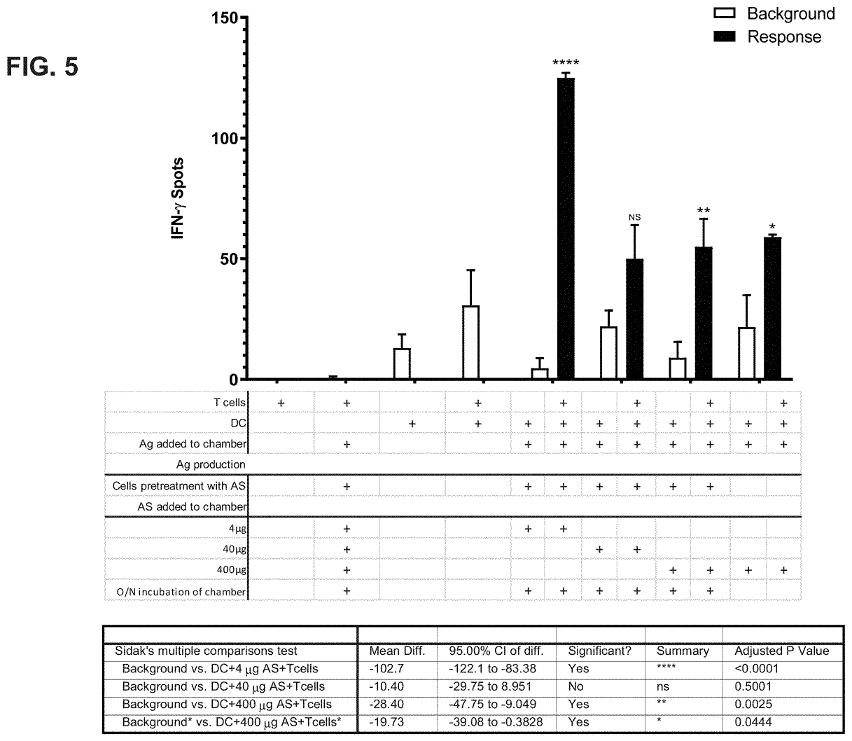

Results: Increased numbers of IFNγ-producing T cells were detected when cultured with dendritic cells pulsed with the contents of chambers containing treated tumor cells indicating that cognate antigens (cross-reactive between individuals) had been produced in the chambers. See FIG. 5. These data indicate that enhanced neoantigen production is obtained by pre-treating human GBM cells overnight with NOBEL, followed by incubation with 4-400 μg, particularly 4 μg, of NOBEL in the irradiated chamber. These data also suggest that high antisense concentration in the chamber may obviate the need for overnight treatment. While 40 μg of NOBEL was effective to induce neoantigen production in FIG. 1, in this experiment, we note that there was a high background response for the cells treated with 40 μg NOBEL sample, meaning that the result did not achieve statistical significance (marked “NS” in FIG. 5).

What is claimed is:

1. A method of producing neoantigens, comprising:
(a) contacting tumor cells from a first subject with a first antisense oligonucleotide to produce neoantigens, wherein the culture is irradiated before or after step (a) and wherein the first antisense oligonucleotide is an insulin-like growth factor 1 receptor (IGF-1R) antisense oligodeoxynucleotide; and
(b) recovering the neoantigens.

2. The method of claim 1, comprising contacting tumor cells with a second antisense oligonucleotide before step (a).

3. The method of claim 2, wherein the second antisense oligonucleotide is the same as the first antisense oligonucleotide.

4. The method of claim 2, wherein tumor cells are contacted with an amount of the second antisense oligonucleotide which is greater than the amount of the first antisense oligonucleotide.

5. The method of claim 1, wherein the tumor cells are selected from the group consisting of tumor cells expressing the insulin-like growth factor 1 receptor (IGF-1R), glioma cells, glioblastoma cells, astrocytoma cells, hepatocarcinoma cells, breast cancer cells, head and neck squamous cell cancer cells, lung cancer cells, liver cancer cells, renal cell carcinoma cells, hepatocellular carcinoma cells, gall bladder cancer cells, classical Hodgkin's lymphoma cells, esophageal cancer cells, uterine cancer cells, rectal cancer cells, thyroid cancer cells, melanoma cells, colorectal cancer cells, prostate cancer cells, ovarian cancer cells, bone cancer cells, smooth muscle cells, pancreatic cancer cells, and any combination thereof.

6.-7. (canceled)

8. The method of claim 1, wherein the culture is in a biodiffusion chamber or a ring-shaped biodiffusion chamber.

9. (canceled)

10. The method of claim 8, wherein the biodiffusion chamber comprises a cell-impermeable membrane.

11. The method of claim 10, wherein the membrane comprises pores with a diameter in the range of about 0.25 μm or smaller or pores with a diameter in the range of about 0.1 μm.

12. (canceled)

13. The method of claim 1, wherein the first antisense oligonucleotide consists of the amino acid sequence of SEQ ID NO: 2 and wherein the nucleic acid backbone of the first antisense oligonucleotide comprises at least one phosphorothioate linkage.

14. (canceled)

15. The method of claim 1, wherein the culture is irradiated with about 5Gy to about 100Gy radiation, preferably about 20 Gy to about 100 Gy.

16. An immunogenic composition comprising the neoantigens produced by the method of claim 1.

17. An immunogenic composition comprising the neoantigens produced by the method of claim 11.

18. The immunogenic composition of claim 17, wherein the volume of the neoantigens does not permit passage of the neoantigens through the pores.

19. (canceled)

20. A method of inducing an anti-tumor immune response, inducing resistance to growth of a cancer, inducing regression of a cancer, or treating a cancer in a second subject in need thereof, comprising administering to the second subject a therapeutically effective amount of the neoantigens produced by the method of claim 1 or an immunogenic composition thereof.

21. The method of claim 20, wherein the induction of the immune response immunizes the second subject against the development of a cancer.

22.-24. (canceled)

25. The method of claim 21, wherein the cancer is selected from the group consisting of glioma, glioblastoma, astrocytoma, hepatocarcinoma, breast cancer, head and neck squamous cell cancer, lung cancer, liver cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, bone cancer, smooth muscle and pancreatic cancer.

26. (canceled)

27. The method of claim 20, wherein the neoantigens or the immunogenic composition thereof are not administered in a device.

28. (canceled)

29. The method of claim 20, wherein the neoantigens or the immunogenic composition thereof are administered to the subject systemically.

30. The methods of claim 20, comprising administering to the subject a follow-on boost dose of the neoantigens or the immunogenic composition thereof.

31. The method of claim 20, wherein
a) the first subject and the second subject are the same;
b) the first subject and the second subject are different.

32.-56. (canceled)

57. The method of claim 1, wherein recovering the neoantigens comprises one or more of the following steps: separating the neoantigens from whole cells by low speed centrifugation, separating neoantigens from cell debris from high speed centrifugation, and chromatographic techniques.


SEQUENCE LISTING (15 sequences)

SEQ ID NO: 1 - DNA, 4099 bp, Homo sapiens
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggcccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtgcaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 2 - DNA, 18 bp, Artificial Sequence
  NOBEL phosphorothioate AS ODN
  misc_feature (1)..(18): joined by a phosphorothioate linkage
tcctccggagccagactt

SEQ ID NO: 3 - DNA, 17 bp, Artificial Sequence
  Recombinant Insulin-like Growth Factor Receptor (IGF-1R) antisense oligodeoxynucleotide (AS-ODN)
ttctccactcgtcggcc

SEQ ID NO: 4 - DNA, 18 bp, Artificial Sequence
  Recombinant Insulin-like Growth Factor Receptor (IGF-1R) antisense oligodeoxynucleotide (AS-ODN)
acaggccgtgtcgttgtc

SEQ ID NO: 5 - DNA, 18 bp, Artificial Sequence
  Recombinant Insulin-like Growth Factor Receptor (IGF-1R) antisense oligodeoxynucleotide (AS-ODN)
gcactcgccgtcgtggat

SEQ ID NO: 6 - DNA, 18 bp, Artificial Sequence
  Recombinant Insulin-like Growth Factor Receptor (IGF-1R) antisense oligodeoxynucleotide (AS-ODN)
cggatatggtcgttctcc

SEQ ID NO: 7 - DNA, 18 bp, Artificial Sequence
  Recombinant Insulin-like Growth Factor Receptor (IGF-1R) antisense oligodeoxynucleotide (AS-ODN)
tctcagcctcgtggttgc

SEQ ID NO: 8 - DNA, 18 bp, Artificial Sequence
  Recombinant Insulin-like Growth Factor Receptor (IGF-1R) antisense oligodeoxynucleotide (AS-ODN)
ttgcggcctcgttcactg

SEQ ID NO: 9 - DNA, 18 bp, Artificial Sequence
  Recombinant Insulin-like Growth Factor Receptor (IGF-1R) antisense oligodeoxynucleotide (AS-ODN)
aagcttcgttgagaaact

SEQ ID NO: 10 - DNA, 18 bp, Artificial Sequence
  Recombinant Insulin-like Growth Factor Receptor (IGF-1R) antisense oligodeoxynucleotide (AS-ODN)
ggacttgctcgttggaca

SEQ ID NO: 11 - DNA, 18 bp, Artificial Sequence
  Recombinant Insulin-like Growth Factor Receptor (IGF-1R) antisense oligodeoxynucleotide (AS-ODN)
ggctgtctctcgtcgaag

SEQ ID NO: 12 - DNA, 20 bp, Artificial Sequence
  Recombinant Insulin-like Growth Factor Receptor (IGF-1R) antisense oligodeoxynucleotide (AS-ODN)
cagatttctccactcgtcgg

SEQ ID NO: 13 - DNA, 17 bp, Artificial Sequence
  Recombinant Insulin-like Growth Factor Receptor (IGF-1R) antisense oligodeoxynucleotide (AS-ODN)
ccggagccagacttcat

SEQ ID NO: 14 - DNA, 20 bp, Artificial Sequence
  Recombinant Insulin-like Growth Factor Receptor (IGF-1R) antisense oligodeoxynucleotide (AS-ODN)
ctgctcctcctctaggatga

SEQ ID NO: 15 - DNA, 15 bp, Artificial Sequence
  Recombinant Insulin-like Growth Factor Receptor (IGF-1R) antisense oligodeoxynucleotide (AS-ODN)
ccctcctccggagcc